SEQUENCE LISTING (13 sequences)

SEQ ID NO: 1 - protein, 534 aa, Arabidopsis thaliana
  translation of SEQ ID NO 2
MATKLDTSSLLLALLSKCSLLTQTNLALSLLVASLASLALSLFFWSHPGGPAWGKYFLHRRRQTTVIPGPRGLPFVGSMSLMSNTLAHRCIAATAEKFRAERLMAFSLGETRVIVTCNPDVAKEILNSPVFADRPVKESAYSLMFNRAIGFAPYGVYWRTLRKIASNHLFSPKQIKRSETQRSVIANQIVKCLTKQSNTKGLCFARDLIKTASLNNMMCSVFGKEYELEEEHEEVSELRELVEEGYDLLGTLNWTDHLPWLSEFDPQRIRSRCSNLVPKVNRFVNRIISDHREQTRDSPSDFVDVLLSLDGPDKLSDPDIIAVLWEMIFRGTDTVAVLIEWILARMVLHPDIQSTVHNELDQIVGRSRAVEESDVVSLVYLTAVVKEVLRLHPPGPLLSWARLAITDTIIDGRRVPAGTTAMVNMWAIAHDPHVWENPLEFKPERFVAKEGEVEFSVLGSDLRLAPFGSGRRVCPGKNLGLTTVTFWTATLLHEFEWLTPSDEKTVDLSEKLRLSCEMANPLAAKLRPRRSFSV

SEQ ID NO: 2 - DNA, 1902 bp, Arabidopsis thaliana
  cDNA
cctttcgtcttaaaccccacagcaaaaagccactctcctttctctctctctcttgttcctctgcatccatggccaccaagctcgacaccagtagcttacttttggccctcttgtccaaatgtagcctccttactcaaaccaatcttgctctctctctcctcgtagcctccctagcttctctcgctctttctctcttcttctggtctcatcccggaggacccgcatggggaaagtacttcctccaccgccgccgtcaaaccaccgtgatacccgggccaagaggcttaccttttgtcggaagcatgtctctcatgtcaaacactctggctcaccgttgcatagccgcaaccgcagagaaatttagagccgaacggttaatggcgtttagtttgggagaaactcgcgtgatcgtcacgtgcaatcctgatgtagctaaagagattctaaacagtccggttttcgctgaccgcccggttaaggaatcagcttattccctcatgtttaaccgtgctatcggtttcgctccttacggcgtttactggcgaaccttgagaaaaatcgcgtctaatcatcttttcagcccgaaacagattaaacgttccgaaacgcagagaagcgtgatcgcgaatcaaatcgtgaagtgtctcacaaaacagagtaacaccaaaggtctctgtttcgcacgtgacttgatcaaaacggcatcgcttaataacatgatgtgctctgttttcggaaaagaatacgagcttgaggaagagcatgaagaagtgagtgagctacgtgaattggtggaagaaggttatgatttactcggtacactgaattggaccgatcatctcccatggctctctgaatttgatcctcaaagaatccggtctagatgctctaatctcgtcccaaaagtaaaccggtttgtgaaccggattatctctgaccaccgtgaacaaactcgtgactcaccgagtgacttcgttgacgtattgctctctctcgatggtcctgataaattatccgaccctgatatcatcgccgttctatgggaaatgatattcagaggaactgacacggtggctgttttgatcgagtggattcttgctaggatggtccttcatccagatattcaatcgacggttcacaatgagcttgatcaaatcgtgggacgatcaagggctgtcgaagagtctgacgtggtgtctctagtatatctaacggctgtggtgaaagaagtcttgaggcttcacccgccaggcccactactctcatgggcccgtttagcaatcacagacacgatcatcgacggtcgtcgtgttccggcggggaccaccgcaatggtgaacatgtgggctattgcacacgatccacacgtgtgggagaatccgttggagtttaaacccgaacgttttgtagccaaggaaggtgaggttgagttctcggttcttgggtctgatttgaggcttgcaccgttcgggtccggtcgtcgggtttgccccgggaagaatcttggtttgaccaccgtgacgttttggactgcgacgcttttgcatgagtttgaatggctgacgccgtccgatgagaagaccgttgacttgtccgagaaactgaggctctcgtgtgagatggctaatcctcttgctgctaaattacgccccaggcgcagttttagtgtatgataagggtaaggctatacacagatacagtggtaactaaagcggaaggaaaattagtgtgaatttaaagcaaaagaataaaataaagaacaaagaaagtaaaggaaacaaaaaaaaagaatcatacaaaaaatactaataagaatggtaatgaagcttttatattaaactaacatcttgatacgtgttgtatatatgatgaaaacattaatgtcacaaagaaaagttaata

SEQ ID NO: 3 - DNA, 7869 bp, Arabidopsis thaliana
  genomic
gaattcgatatctagatccgaaactatcagtatcagtatttacagtgtatggcaatgtagtggatatatatagatttaaaatttattcattgatctattccggatgagtagagaatacatatcggtcgtttaaaatgatattaatttgtagtttacataacaatagatgtggacaagccattgttgaagtccttgattgcccccacgtcttcgatgataaaataggtcgaaagggtcaacaacaggttagttaacgttaaaactaatttaactcttggaatagtatattcagttaaatttaaaataaagtatatctcgttttataataagacgtacagttgatacttatattcaagagaaaattaaataaattgctgatttagtttacattcaacttgaacattctattaaccagatattgttgtattctctgtatattattattacgttatatcatatatgcttagtatcttctaaaaatcccctgattcggttgacttcgatcatacatatatcgattatcgagcagtaacacaaagcatattataaaagagctcttcgaatatgacgtcatccgaagaatcatagatcacacaaagcgtaagtgtcacgcgccctaacgtaacctgccattcagttcacatccctcgcgcatggcggcgctaaaccgttaaatctcattttgccaacaacaacaactctaccgttataatttgtcttttaaattatacgtttacactttcttgttactattggatgacatattgttgattagtttaatagatagttaacattaacaaaggaaagataactacactccttattacgagtgaaagttaaaatatacataaaaaacgttacgatcatggactgtatttaacaaagatattattcttaaatgtccaacacacttactgttagtaaataaatataataatatatagtgctgcaagcctgcaaccgtgcagcatacagattaaatcaattattttttcttataaaataatcatcaagtattaaaatattagaacttgatgtcgtatgctgtgatcgaatatgtacttaactgcaccgagactaacgcatatgcactgcaagtaaaagtaaccgttgtttttaagttttaacagtactctcagttggaattaattccataacaaagctatgacgttaaatctataataaaagacatttttacaaaaatttacgaatatagatatatagtttaccagttaaatcagctaactacgattacaattaacaaattcaatgaattcacaatagtgggtctatcaagtttcatcagttttatttaacggccacgagaagttatgttaattgctatatgcattactgccattgcatttacacctcttgatcttacgtttttaactttgtaactgactttgtcaataatttttcgaaatgaagattaattttacattcaaatccatctcatggtatgaaaaaaaaaattcccggagcttacgtttcgttttataaaaagatatgttttgtaagtggtccgaaatcttaatcacttttaatttttgcacttttatatttgatgtgtatttgtctactagattggtgaataaagagcatattaattcattgcatttaatcaactttttttcttgtggctgcaaggccttgctttttttaaaaaatatcctgaaataaatttggtttatgtttaatcaaataaaataattatcagaacccacataataatacaagattttcggtaaacttcaaataaataaaggttttaaaaactttttttcggagaaaaactacaagttcaaattttatgttacaacaaggaagcaagcaaattcaaacttgcccaagtgtatcaagttaaaagtacaatattaaggtaagaagaatgaaatacacacaaaaaatatagtgtttaatataaaaaacaacatttccttcactctttcagttatataataataatacctattataaatgaaatataatatagtttttgaactaattacaaaaactaacaagaggggcttctaatgtttttttctatataaatacctgaagcctttcgtcttaaaccccacagcaaaaagccactctcctttctctctctctcttgttcctctgcatccatggccaccaagctcgacaccagtagcttacttttggccctcttgtccaaatgtagcctccttactcaaaccaatcttgctctctctctcctcgtagcctccctagcttctctcgctctttctctcttcttctggtctcatcccggaggacccgcatggggaaagtacttcctccaccgccgccgtcaaaccaccgtgatacccgggccaagaggcttaccttttgtcggaagcatgtctctcatgtcaaacactctggctcaccgttgcatagccgcaaccgcagagaaatttagagccgaacggttaatggcgtttagtttgggagaaactcgcgtgatcgtcacgtgcaatcctgatgtagctaaagagattctaaacagtccggttttcgctgaccgcccggttaaggaatcagcttattccctcatgtttaaccgtgctatcggtttcgctccttacggcgtttactggcgaaccttgagaaaaatcgcgtctaatcatcttttcagcccgaaacagattaaacgttccgaaacgcagagaagcgtgatcgcgaatcaaatcgtgaagtgtctcacaaaacagagtaacaccaaaggtctctgtttcgcacgtgacttgatcaaaacggcatcgcttaataacatgatgtgctctgttttcggaaaagaatacgagcttgaggaagagcatgaagaagtgagtgagctacgtgaattggtggaagaaggttatgatttactcggtacactgaattggaccgatcatctcccatggctctctgaatttgatcctcaaagaatccggtctagatgctctaatctcgtcccaaaagtaaaccggtttgtgaaccggattatctctgaccaccgtgaacaaactcgtgactcaccgagtgacttcgttgacgtattgctctctctcgatggtcctgataaattatccgaccctgatatcatcgccgttctatgggtatgtacacgtggcttgtatttttacttttgtactgaagaaaaaacttacaaaaattcatatttattgtactgtatgcgtttaattacatttatactatgatttttggataattaaatatgccattttaagtaactaaagtattggcaattttgggttttaataacaacaggaaatgatattcagaggaactgacacggtggctgttttgatcgagtggattcttgctaggatggtccttcatccagatattcaatcgacggttcacaatgagcttgatcaaatcgtgggacgatcaagggctgtcgaagagtctgacgtggtgtctctagtatatctaacggctgtggtgaaagaagtcttgaggcttcacccgccaggcccactactctcatgggcccgtttagcaatcacagacacgatcatcgacggtcgtcgtgttccggcggggaccaccgcaatggtgaacatgtgggctattgcacacgatccacacgtgtgggagaatccgttggagtttaaacccgaacgttttgtagccaaggaaggtgaggttgagttctcggttcttgggtctgatttgaggcttgcaccgttcgggtccggtcgtcgggtttgccccgggaagaatcttggtttgaccaccgtgacgttttggactgcgacgcttttgcatgagtttgaatggctgacgccgtccgatgagaagaccgttgacttgtccgagaaactgaggctctcgtgtgagatggctaatcctcttgctgctaaattacgccccaggcgcagttttagtgtatgataagggtaaggctatacacagatacagtggtaactaaagcggaaggaaaattagtgtgaatttaaagcaaaagaataaaataaagaacaaagaaagtaaaggaaacaaaaaaaaagaatcatacaaaaaatactaataagaatggtaatgaagcttttatattaaactaacatcttgatacgtgttgtatatatgatgaaaacattaatgtcacaaagaaaagttaatactatgtgttttttaatatttttttctcgctttcttttggcattctgattttttgttaatctaaatttcaaaacttgatttcctcgtttcatgggaaacgatcttcgcttgaacacaaacaaagccccgattacaagcaaatatactatatcatcctttcatcacagatcagaaaacttaaaacatagtaattaataatgtaattattatttaaattcattttatttcaagaagaaaaaattacaataataaatgttgaatacattaggctataccaattcatcataatgaagcaagttcatacatgtatagaatatatttttgggtaagaaactcaaatcccatgtaaccttaaatttttaaaattgtcgaccgataatgctagcgaacacatatttttcaaaaaaaaaatagccgaataaatcgaatgagttgttttcacattttttttccccagccaaaaatatacaaaaagtttcgtctttattaaagttgcagattcgacatatcatctcccttactgtttattatttgtctactatatgtgtaaagatcaataattacattacaaataaacaaaaaaatataacgtatgtgctgtagatatatttacttaaaactactatatgtatatatggaattcatattttattggttttaaattatttctttctatttttcatatataatattagttgaaatagagtagccagagttctcactcgatataattaatataactttccatgttttttgttttctttaaaaataaaagaaaaactcactcgatataactaaagtactaaacaatcaaattcaataatcaatataattaataaaactcaatttatggtatagtaaacgattaacaacttaatatagagaaaacgaccggcaggcctttatacttgcccaaatctactagacacccattttaagagaatggataataatctcttagacatatatcaagatggagacctcctagtttaaaatgttggccacatggttagtgtgtttgacaaacaaactaaccactatttaaagccttttcaaaaaaaaaaaaaacctaaccactatttactactagtcgatttactaaatatgggaaaagaaaaaaaaaactaattttaaagtaatagtttatataagataatttgactgaaaatattttcaaacttcaaattgtgtgatcgaacataaaaaaataaaaaatcatgataataagagttcttgaatttatgtttatagatacgaaagattgtgacacatgaattattaaaggatggtggtatttttgatccagtgaggagcaggcacataagcattaggaaagaacaaaatatgacaagatttgaactaattgtggtctaatcatatatacaactgagagatcttaaaagtggataagtatctctataaagaaagttttttttatggtggaagcatcttcactggttgctgagaggttttcataatgacgcgacgccatgtatgtttctcaataaaagtgattgcagcaaaagatatgactattttgtttccgcaatttgtagtccaagtgcaaacaaaacatgatttacatgtaaactgaaaattggcttgatcaagaattatcgataaccaatttgggcctatcaacataaataccattgtatctatatacatgggccaaaattgacaaagtatattatttgatagacatttaagatataataaatgtcagtattctttttttttttaggtgagacaaatgtcagtaatctactaatctttgacacaatcatagcgtatccaatttatatttgacacaatcaggtgaaaacaaaggactggtaaaagagtattattgatcgtttttccaccattattccaatatccactctaattgcatgatttttacaattcatcatctacaaacctcaaaaaaagttgaagaatatttgcatatatagagtcacaaactcataaataacacatccaaatattaatatctatggccatttagtgaactcaaagaccttaagaacgatatcaagacctgttgggatttgccaaatgaaaagaagaacatttactgcattcaacgctatatgtagactcctcgccgtatcgttccctttctgcattgccggcaccaatgctgccgccgccgcccacaacaccgttattcctgccaccaccaccaccatgaacacaaaatgtgacacgttttgtacatccaataacaaatataaaccacattttttctcatagttgtgatgaatggtaacttttgaaatggcacaatactatttataccaatataactaaaatccaaagttctgatcaaaacttaaaccacatcagaccaagccaaaacctgactaagcaacccaagattacacactttgataaattagaacctaaaatcaaggaaatgaaaaataagacgagagataggtttaaggtacctgcaccggcgtaaagatgaggaccggggaagagtttaccagtacgaagataagtgttaacaccaccaaagacagcttccaaaacaccgaaacctaacaaaacagagccagcgtcaaagtgtttgtctctgtaagaccctttgaccaactctttcctctcttcagttagccgttggatctgaagctcagtcgtagaaggaggcgacgaagaatcaacggctgtggaaccatcaggggaaacaggagttggtttaagctgtttcttgagatcactaatctcactctgtatcgtacggacacgtctccattgccaacctaagtaaccagtccaaagagtgtatgcaaataaacctcccatcacaattgggtgtatcaacgcaaatgatctcccttctaatattccgaattctcctccagctgctgctgcatccttttgaaattgcaaagaaaacaagaatcttgatcatctggttatatttgtagaatgtgaaactattggggaaagatgtgaaactttttgattacctgtgggtcgaggaagaaaggcaatgcgatgactgcgagagggagagaggatgatttaaggagctgaagagcgttgggagtggagggtatttctggggttttacatggttgtgatttggtggtgattggtttgagagagtttagggttttagggtgatgagtgcgaggaagaagatggagtgtggagagacaatgaagtgtggtcgccatactcgcgattcttggaacagagattgtggcaaaaagagtgaagaagaaacaatagagacatcctaaaccgcaaaaatgatcgaaccaaaacagataagagaactccgttgaatctccacgtgtcagaaaactggacacttacataaactttaattcttttcttcttcaccgacggaaaatgggtctgtatataaacatgtataaatttaattctataagaaaagactccaacgagttcttcggtatggggctaacttattgtgggcctatttataaagaaggcccattatgatttttgaatttccgaatgtacccatgatggtatgacgggcttttttaagtcaaccaggctctgcttttcttcgacgagcgtagtagtcgtcgtcgtcgtctagggtttgtttttcgtttcttctccgattgttcagaggtaacttttctccgataattatacttgctccatgttccatataccttattttatcccttacatgttactaatttcgaagtttattggattcgttaggaaattgcgaattaagaa

SEQ ID NO: 4 - protein, 530 aa, Arabidopsis thaliana
  translation of SEQ ID NO 5
MATKLESSLIFALLSKCSVLSQTNLAFSLLAVTIIWLAISLFLWTYPGGPAWGKYLFGRLISGSYKTGNVIPGPKGFPLVGSMSLMSSTLAHRRIADAAEKFGAKRLMAFSLGETRVIVTCNPDVAKEILNSPVFADRPVKESAYSLMFNRAIGFAPHGVYWRTLRRIASNHLFSTKQIRRAETQRRVISSQMVEFLEKQSSNEPCFVRELLKTASLNNMMCSVFGQEYELEKNHVELREMVEEGYDLLGTLNWTDHLPWLSEFDPQRLRSRCSTLVPKVNRFVSRIISEHRNQTGDLPRDFVDVLLSLHGSDKLSDPDIIAVLWEMIFRGTDTVAVLIEWILARMVLHPDMQSTVQNELDQVVGKSRALDESDLASLPYLTAVVKEVLRLHPPGPLLSWARLAITDTIVDGRLVPAGTTAMVNMWAVSHDPHVWVDPLEFKPERFVAKEGEVEFSVLGSDLRLAPFGSGRRICPGKNLGFTTVMFWTAMMLHEFEWGPSDGNGVDLSEKLRLSCEMANPLPAKLRRRRS

SEQ ID NO: 5 - DNA, 1593 bp, Arabidopsis thaliana
  cDNA
atggctacgaaactcgaaagctccttaatctttgcccttttgtccaaatgcagcgttctaagccaaaccaaccttgccttctccctcctcgccgtcacaatcatctggctcgccatatctctcttcttatggacctatcccggtggacctgcttgggggaaatacctcttcggccggttaatatccggttcatacaaaaccggaaacgttattcccggtccaaaaggcttccctttggttggaagcatgtcactcatgtcaagcactctagctcaccgacgaatcgctgatgcagctgagaaattcggagccaagaggctcatggctttcagcttaggagagactcgcgtgatcgtcacgtgcaatcccgacgtagcgaaagagattctgaatagcccggtttttgctgatcgaccggttaaagaatcggcttactcactgatgtttaacagagcaattggttttgcaccacacggtgtttactggcgaacgcttcgccgtatcgcttcgaaccatctctttagtacaaaacaaatcagaagagccgagacgcaacgacgagtgatctcaagccagatggttgagtttcttgaaaaacagagtagtaacgaaccctgttttgttcgtgagttgcttaaaacggcgtcgcttaacaacatgatgtgctctgtattcggacaagagtatgagcttgaaaaaaaccatgttgagttacgtgaaatggtcgaagaaggttatgatttgctcggaacgttgaattggactgatcaccttccttggctatcggagtttgatcctcaaagactccggtctagatgttccacactcgtaccaaaggtaaaccggtttgtatcccggattatatccgaacaccgtaatcaaaccggtgatttgcctcgtgatttcgtcgacgttttgctctccctccatggttcagataaattatccgacccggacataatcgccgttctttgggagatgatattcagaggaacagacacagttgcggtcttaatcgagtggatcctcgctaggatggtccttcatccagatatgcaatcaacggtacaaaacgagctggatcaagtagtcgggaaatcaagagccctagatgaatctgacttggcttcacttccatatctaacggctgtggtgaaagaagtattgaggcttcatcctccaggcccacttctatcatgggcccgtttggccataacagacacgatcgttgatggtcgtcttgttccggcagggaccacagcaatggtgaacatgtgggccgtatcgcatgatccacacgtgtgggttgatcctttggagtttaaacctgagaggttcgtggcaaaagaaggtgaggtggagttttcggttcttgggtcggatttgagacttgcacctttcgggtcgggtcgtcggatttgccccgggaagaatcttggttttactaccgttatgttttggacggcgatgatgttacatgagtttgaatggggaccgtccgatggtaacggcgttgacttatctgagaaactgaggctttcttgcgagatggctaatcctcttcctgctaaattgcgccgtaggcgcagttaa

SEQ ID NO: 6 - protein, 523 aa, Glycine max
  translation of SEQ ID NO 7
MTSHIDDNLWIIALTSKCTQENLAWVLLIMGSLWLTMTFYYWSHPGGPAWGKYYTYSPPLSIIPGPKGFPLIGSMGLMTSLAHHRIAAAAATCRAKRLMAFSLGDTRVIVTCHPDVAKEILNSSVFADRPVKESAYSLMFNRAIGFASYGVYWRSLRRIASNHLFCPRQIKASELQRSQIAAQMVHILNNKRHRSLRVRQVLKKASLSNMMCSVFGQEYKLHDPNSGMEDLGILVDQGYDLLGLFNWADHLPFLAHFDAQNIRFRCSNLVPMVNRFVGTIIAEHRASKTETNRDFVDVLLSLPEPDQLSDSDMIAVLWEMIFRGTDTVAVLIEWILARMALHPHVQSKVQEELDAVVGKARAVAEDDVAVMTYLPAVVKEVLRLHPPGPLLSWARLSINDTTIDGYHVPAGTTAMVNTWAICRDPHVWKDPLEFMPERFVTAGGDAEFSILGSDPRLAPFGSGRRACPGKTLGWATVNFWVASLLHEFEWVPSDEKGVDLTEVLKLSSEMANPLTVKVRPRRG

SEQ ID NO: 7 - DNA, 1611 bp, Glycine max
  cDNA
aagcactatccctcccaccatgacaagccacattgacgacaacctctggataatagccctgacctcgaaatgcacccaagaaaaccttgcatgggtccttttgatcatgggctcactctggttaaccatgactttctattactggtcacaccccggtggtcctgcctggggcaagtactacacctactctccccccctttcaatcattcccggtcccaaaggcttccctcttattggaagcatgggcctcatgacttccctggcccatcaccgtatcgcagccgcggccgccacatgcagagccaagcgcctcatggcctttagtctcggcgacacacgtgtcatcgtcacgtgccaccccgacgtggccaaggagattctcaacagctccgtcttcgccgatcgtcccgtcaaagaatccgcatacagcctcatgtttaaccgcgccatcggcttcgcctcttacggagtttactggcgaagcctcaggagaatcgcctctaatcacctcttctgcccccgccagataaaagcctctgagctccaacgctctcaaatcgccgcccaaatggttcacatcctaaataacaagcgccaccgcagcttacgtgttcgccaagtgctgaaaaaggcttcgctcagtaacatgatgtgctccgtgtttggacaagagtataagctgcacgacccaaacagcggaatggaagaccttggaatattagtggaccaaggttatgacctgttgggcctgtttaattgggccgaccaccttccttttcttgcacatttcgacgcccaaaatatccggttcaggtgctccaacctcgtccccatggtgaaccgtttcgtcggcacaatcatcgctgaacaccgagctagtaaaaccgaaaccaatcgtgattttgttgacgtcttgctctctctcccggaacctgatcaattatcagactccgacatgatcgctgtactttgggaaatgatattcagaggaacggacacggtagcggttttgatagagtggatactcgcgaggatggcgcttcatcctcatgtgcagtccaaagttcaagaggagctagatgcagttgtcggaaaagcacgcgccgtcgcagaggatgacgtggcagtgatgacgtacctaccagcggtggtgaaggaggtgctgcggctgcacccgccgggcccacttctatcatgggcccgcttgtccatcaatgatacgaccattgatgggtatcacgtacctgcggggaccactgctatggtcaacacgtgggctatttgcagggacccacacgtgtggaaggacccactcgaatttatgcccgagaggtttgtcactgcgggtggagatgccgaattttcgatactcgggtcggatccaagacttgctccatttgggtcgggtaggagagcgtgcccagggaagactcttggatgggctacggtgaacttttgggtggcgtcgctcttgcatgagttcgaatgggtaccgtctgatgagaagggtgttgatctgacggaggtgctgaagctctctagtgaaatggctaaccctctcaccgtcaaagtgcgccccaggcgtggataagagagagttgaagcttttat

SEQ ID NO: 8 - protein, 553 aa, Pinus radiata
  translation of SEQ ID NO 9
MENRRSSGGSGWWVCVLPLFTKDGPAYFLHSSSDDVSAWRQWPLYIALLIVAVCAVLVSWLSPGGCAWAGRHKRGRVAIPGPKGWPIIGSLMDMSVGLPHRKLESLARLHGAKQLMSFSLGCTPAVITSDPEVARELLTSPHFANRPLKQSAQQLLFGRAIGFAPNGGYWRLLRRIASAHLFAPRRIAAHEAGRQADVVAMLDDIQKEYHSKGVVRVRRHLQGAALNNIMGSVFGRRFDMSHENEEVKKLREMVDEGFQLLGAFNWADHLPWLRPLDPLRIHARCARLVPRVTTFVSNIIEQHRREEQRRESGDQCDFVDVLLSLQGEDKLDEEDMIAVLWEMIFRGTDTTALLTEWTMAELVLHPEAQKKAQAELDAVVGHDRSVKDSDIPKLPYIQAVVKEALRMHPPGPLLSWARLSTEDVNMGDGMCVPAGTTAMVNMWSITHDPNIWESPYEFRPERFVVFEGGEEVDVRGNDLRLAPFGAGRRVCPGKALGLATVNLWVAKLLHHFEWLPHAEHPVDLSEVLKLSCEMARPLHCVPVTRVPFAKFSD

SEQ ID NO: 9 - DNA, 1990 bp, Pinus radiata
  cDNA
cttcctggttttgctactgctcgctgtttccttgtcgctttggtttctctgtttgaaaggccatttctaaaaatggagaatcgcagaagctctggaggaagcgggtggtgggtctgtgtccttccgctcttcacaaaagatggccctgcttacttcttgcactcctcatctgacgatgtttctgcatggcgtcagtggcctctgtatattgcactgcttattgttgccgtctgtgctgttctagtctcctggttgagccctggaggttgtgcgtgggctggacggcataagagaggccgcgttgccattcctggccccaaaggatggccgattattggaagtctaatggacatgagcgtcggccttcctcaccgcaagcttgaatctctcgctcgtcttcatggtgcgaagcagctcatgtcctttagcttgggctgcacgcccgctgttattacgtcggatcccgaggtggcccgagagttgctcacctcccctcacttcgccaacagacccctcaagcaatccgcacagcagttgctgtttgggagggctattggcttcgcacccaacgggggctactggaggctgctcaggagaattgcttctgctcatctctttgctcctcgcaggatcgctgcccatgaagctggccgccaggccgacgttgtggcaatgctggatgatatccaaaaggagtatcattctaagggtgttgttagggtaagacgacacctgcaaggagcggcgctgaacaatatcatgggaagcgtctttggaaggaggttcgacatgtctcatgagaatgaagaagtgaagaagctgcgggagatggttgatgaagggttccaactgttaggagcgttcaactgggcggatcatctcccatggctgcgaccgctggaccctcttcgaatccatgctcgctgcgctcgcctcgtaccgcgtgtgacaactttcgttagcaatatcatcgaacagcaccgtcgggaagagcagcgtagagaaagcggcgatcaatgtgattttgttgacgttctgctgtcgttgcaaggagaggataagctcgacgaggaggatatgatcgctgttctctgggagatgatctttcgtggcactgatacgacggctctgctgacagagtggaccatggcagaactggttctgcaccctgaagctcagaaaaaagctcaggcagagttggacgccgtagtggggcacgaccgaagcgtgaaggattcagacattccaaagctgccgtacatccaagcagtggtgaaagaggcgctgcgaatgcatccacctggaccgctcctatcatgggctcgtctctctacagaagatgtcaacatgggcgatggaatgtgtgttccagcgggcacaactgccatggtgaatatgtggtcgattactcacgaccccaacatttgggaatcgccatatgagttccgtccggagaggtttgtggtgtttgagggcggggaagaggtggatgtaaggggcaacgatctgaggcttgcgccttttggtgcgggtcggcgagtatgccccggaaaggctctgggccttgccactgttaatctctgggttgcgaagttgctccatcattttgaatggcttccacatgctgaacatccggttgatctgtcggaggtccttaagctgtcatgtgaaatggctcgtcctcttcattgcgttccagtcacccgggtgcctttcgctaaattttcggattagctaatggccattatcatccaaatcgtgatgtttttttgggtagtatttgtaggcattacgcaatgccgttcagtttttgcctgcgattatgtatttgagcttacgaatggtagattttccgacctgtccttgaaaggggcttatcatcctcactctgttctcattctcagggcacctttttgttattaatggtgttaaatggagtgtttatgtttttgggctttgatccgtctgcttaatctatatgaatcaaatc

SEQ ID NO: 10 - protein, 547 aa, Zea mays
  translation of SEQ ID NO 11
MAMASAACSCTDGTWWVYALPALLGSDTLCAHPALLAGLIFLATVSVALLAWATSPGGPAWTNGRGASASLLSWDPVVCPCSAASSRCPGAAAPRPRRDGPRRRPRAKELMAFSVGDTPAVVSSCPATAREVLAHPSFADRPVKRSARELMFARAIGFAPNGEYWRRLRRVASTHLFSPRRVASHEPGRQGDAEAMLRSIAAEQSASGAVALRPHLQAAALNNIMGSVFGTRYDVTSGAGAAEAEHLKSMVREGFELLGAFNWSDHLPWLAHLYDPSNVTRRCAALVPRVQTFVRGVIDEHRRRRQNSAALNDNADFVDVLLSLEGDEKLGDDDMVAILWEMVFRGTDTTALLTEWCMAELVRHPAVQARVRAEVDAAVGAGGCPTDADVARMPYLQAVVKETLRAHPPGPLLSWARLATADVPLCNGMVVPAGTTAMVNMWAITHDAAVWADPDAFAPERFLPSEGGADVDVRGVDLRLAPFGAGRRVCPGKNLGLTTVGLWVARLVHAFQWALPDGAAAVCLDEVLKLSLEMKTPLVAAAIPRTA

SEQ ID NO: 11 - DNA, 2087 bp, Zea mays
  cDNA
ctaggtagcaccgtagcagctactagaagcagctagccagaacaactcgtccatggcgatggcctccgcggcttgctcatgcacggacggcacgtggtgggtgtacgcgctcccggcgctgctcggctccgacaccctgtgcgcccacccggccctcctggctggcctgatctttctggccaccgtctcggtggctctgctggcgtgggccacgtcgccgggcggtccggcgtggacgaacggccgcggcgcctcggcgtcactcctatcgtgggaccccgtggtctgcccgtgttcggcagcatcttcgcgctgtcccggggctgccgcaccgcgccctcgccgagatggcccgcgccgcaggccccgggccaaggagctcatggcgttctccgtcggtgacacgcccgcggtcgtgtcgtcctgcccggccacggcacgtgaggtgctcgcgcacccgtcattcgccgaccgccctgtgaagcggtcggcccgggagctcatgttcgcgcgtgccatcgggttcgcgcccaacggcgagtactggcgccgcctccgccgcgtcgcgtccacgcacctattctccccgcgccgggtcgcctcgcacgagccgggacgccaaggtgacgcggaggccatgctccgctccatcgccgccgaacagtcggcctctggcgccgtcgccctccgcccgcacctccaggccgccgctctcaacaacatcatgggcagcgtcttcggcacgcggtacgacgtcacatcaggcgccggcgccgcggaggccgagcatctcaagagcatggtgcgcgaggggttcgagctcctcggcgccttcaactggtccgaccacctcccctggctcgcccacctgtacgacccaagcaacgtcacccgccggtgcgccgcgctcgtgccgcgcgtccagaccttcgtccgtggcgtcatcgacgagcaccggcgccgccgccaaaactccgccgccctcaacgacaatgctgacttcgtcgacgtgctcctctccctcgagggtgacgagaagctcggcgacgacgacatggtcgccatcctctgggagatggtcttccgcggtacggacacgacggcgcttctgaccgagtggtgcatggcggagctggtgcgccacccggcggtgcaggcgagggtgcgcgccgaggtcgacgcggctgtcggtgccggaggttgccccaccgacgccgacgtggcgcgcatgccgtacctgcaggcggttgtgaaggagacgctgcgcgcccacccgcctggcccgctgctgagctgggctcgcctcgccaccgccgacgtgccactctgcaacggcatggtggtcccggctggcaccacggcgatggtgaatatgtgggccataacccacgatgccgccgtgtgggccgacccggacgcgttcgcgccggagcggttcctgccctccgagggcggcgccgacgtggacgtccgcggcgtcgacctccgcctggccccgttcggcgccgggcgtcgcgtctgccccggcaagaacctgggcctcaccaccgtgggcctctgggttgcccgcctcgtgcacgccttccagtgggccctgcctgacggcgcggcggccgtttgcctcgacgaggtcctcaagctctccctggagatgaagacgccgctcgtcgccgcagccatcccccgcaccgcctgatccgtcctgccgccgacgcgtcacgtcacgcgttgtttgcatggatgatggtatctttgtctgtctgtgtggtcttcgctaaagtttgcttcttctcgatcgtcggttcgttcgtgcctccaccttagcctagggtttggtttcttgcaaggtagtgagtgtgtcttagtctcaccatcaccggggctccaattttggaaagctgcgtgttaggagttaacccctagacatgtttgcgtcttgatcgccaccacccatcagtatcagcgcagaaactacatatagatcagtgtttgtcgaccagtcatggaagtcgtgtgctctcaagtctgatggtattatatacatatatatgtattgtaatgtgattatcaagaaccgtgctatttac

SEQ ID NO: 12 - protein, 426 aa, Phalaenopsis sp.
  translation of SEQ ID NO 13
MAFSVGLTRFIVSSHPKTAKEILSSPAFADRPIKESAYELLFNRAMGFAPFGDYWRNLRRISSTYLFSPRRVSSFEKQRSEIGEGMVRDMKRMMERNGVVEVRRMLHYGSLNNIMLTVFGKKFDFAKDEGLELELILKEGYELLGIFNWGDHLPLLGWLDLQGVRRRCRTLVAKVNVFVKKIIDEHKRRANGVGIDEGEGEDFVDVLLGLEEKDRLSESDMVAVLWEMIFRGTDTVAILLEWTLARMVLHPDIQSKAQVEIDSVVDSSRPVLDSDIQRLPYLQSIVKETLRMHPPGPLLSWARLAIHDVPVDGHMIPAGTTAMVNMWAITHDECNWAEPNKFNPDRFIDEDVNILGSDLRLAPFGSGKRVCPGKTMALAAVHLWLAQLLKSFKLLPSRNGVDLSECLKMSLEMKNPLVCVAVPRFE

SEQ ID NO: 13 - DNA, 1799 bp, Phalaenopsis sp.
  cDNA
cggcaccactcctctctgttcctctaatatctggttaaaaatgacaatgtcatccatggattcatcttcaataatattaacttatctctccccaacactttctccagctatcgccgcttctatcatcatcatctcagctctactactctttcccggcggtctggcgtgggccctttccctcaagcgcccaacattctccgggcccaccggaattgtttttgctctcgccagctctgctgctcataagtcacttgccgccctagctcgctccgttcgacgccctccgcctcatggctttctcggtcggcctcactcgcttcatcgtttcaagccacccgaaaaccgcaaaagagattctttcaagcccagccttcgctgatcggcccattaaagaatcagcatacgaacttctgtttaatcgcgctatgggttttgccccatttggggattactggagaaacctgagaaggatttcgtccacatatcttttcagtccgcggcgagtttcatcgttcgagaagcaacggagtgagattggcgaaggaatggtgcgggatatgaaaagaatgatggagagaaatggagttgtagaagtgaggagaatgttgcactacgggtctttgaataacatcatgttgactgtttttgggaaaaagtttgattttgcaaaggatgaggggttggagcttgagttgatccttaaggaaggatatgagttacttgggatcttcaactggggtgatcatttgcctcttttgggatggttagatttgcaaggtgtgaggagaagatgcagaacacttgtggctaaggtcaatgtatttgtgaagaagatcatagacgagcataagaggagagccaacggcgtagggattgatgagggtgaaggtgaagattttgttgatgtgcttcttggtttggaggagaaagatagactctcagaatctgatatggtcgcagttctttgggaaatgatctttagaggaactgatactgttgccatcctattggaatggacgttggctagaatggttcttcatcctgatattcaatcgaaggcacaagttgagattgattctgtcgttgactcttcaaggccagtattggattctgatatccaacgacttccttatctccaatctatagtaaaagaaacccttcgaatgcatcctcctgggcctctattgtcatgggctcgcctagctatccatgacgttcctgttgatggtcacatgattcctgctgggacgactgcaatggtgaacatgtgggcaataacacatgacgaatgcaactgggctgagcctaacaaattcaatcctgatcgattcatcgatgaagatgtcaatattcttggttccgatttaaggttggcaccctttggctccggtaaaagagtttgccctggcaaaacgatggcattggctgcagttcatctttggttggctcagttgctgaaaagcttcaaattgcttccttcgagaaatggtgtagatttgtctgagtgcctaaagatgtctctcgagatgaagaatcctttggtatgtgtggctgttccaaggttcgagtagtcctgctaagatgacgtctagttataagaaatttgttctttgcaaattgtggccaacataaatgatttcgtaagctagcaacttatggataatgtcggtacatgttcgtttaaagtgtcaactttgtttggttgaattttaaaatttgacattgtaataaagattctctggttctatgtaaatattgtaattcagcttataatataagaaagaaatgaatttgttgct

(19) United States
(12) Patent Application Publication
(10) Pub. No.: US 2002/0152495 A1
(43) Pub. Date: Oct. 17, 2002

(54) PLANTS HAVING SEEDLESS FRUIT

(76) Inventors: TOSHIRO ITO, PASADENA, CA (US); MICHAEL FROMM, KENSINGTON, CA (US); ELLIOT MEYEROWITZ, PASADENA, CA (US)

Correspondence Address: GRAY CARY WARE & FREIDENRICH LLP, LISA A. HAILE, J.D., PhD, 4365 EXECUTIVE DRIVE, SUITE 1100, SAN DIEGO, CA, 92121-2133, US

(21) Appl. No.: 09/349,385

(22) Filed: Jul. 9, 1999

Related U.S. Application Data
(60) Provisional application No. 60/115,967, filed on Jan. 15, 1999.

(51) Int. Cl. C12N 15/82; C12N 15/87

(52) U.S. Cl. 800/278

(57) ABSTRACT

The present invention provides compositions and methods for affecting the development of fruit in a plant. In particular the invention provides compositions and methods for producing a plant with parthenocarpic fruit and/or enlarged fruit.

### FIELD OF THE INVENTION

This invention relates to compositions and methods for producing plants with seedless fruit or enlarged fruit.

### BACKGROUND OF THE INVENTION

Most fruit crops have fruits that will only develop in the presence of seeds. However, there is a need to produce seedless fruits, because seedless fruits have an increased edible portion as the seed cavity may be smaller or greatly reduced, the fruit is sweeter or fleshier, and to satisfy consumer preferences.

At the present time seedless fruits may be produced by topical application of plant hormones, such as auxin analogues (in the case of strawberry or tomatoes) or gibberellins (in the case of apples, currants, grapes, cucumbers or eggplants) (Lippari et al. (1988) Acta Hort. 229:307-312). Also, a breeding technique exists for generating seedless fruits which entails generating triploid plants. The triploid plants form abnormal embryos and normal ovular development into seed is terminated prematurely. This breeding technique is currently in use for generating seedless watermelons (Kihara (1951) Proc. Amer. Soc. Hort. Sci. 58:217-230). Other possible methods for generating seedless fruit are described in U.S. Pat. No. 5,877,400 where is described a gene encoding a plant hormone, a plant hormone precursor or an enzyme in a plant hormone biosynthetic pathway which is temporally expressed to inhibit seed production or in U.S. Pat. No. 5,773,697 where is described one or more cytotoxic genes temporally expressed in seeds at a time such that fruit maturation is normal but seed maturation is decreased.

The present invention provides compositions and methods for producing plants with seedless fruit or enlarged fruit.

### SUMMARY OF THE INVENTION

In one aspect, the invention provides an isolated polynucleotide comprising a nucleotide sequence encoding a cytochrome P450 polypeptide which when expressed in a plant produces a plant with at least one phenotype selected from the group consisting of parthenocarpic fruit and enlarged fruit. In one embodiment, the encoded P450 polypeptide may comprise (a) SEQ ID No. 1; (b) a sequence comprising conservative substitutions to SEQ ID No. 1; (c) a homologous sequence which when expressed in a plant produces parthenocarpic fruit or enlarged fruit; or (d) a sequence comprising a fragment of (a), (b) or (c). Preferably, when these sequences are expressed in a plant parthenocarpic fruit or enlarged fruit are produced. In a second embodiment, the encoded cytochrome P450 polypeptide comprises amino acids 301-309 of SEQ ID No. 1, 319-336 of SEQ ID No. 1, 385-405 of SEQ ID No. 1, 415-427 of SEQ ID No. 1 or 463-477 of SEQ ID No. 1 or combinations of these sequences. In a third embodiment, the nucleotide sequence comprises the sequence of SEQ ID No. 2 or 3, a homologous sequence or a fragment thereof.

Additionally, the polynucleotide sequence may comprise a tissue-specific or active promoter, particularly, a promoter active in fruit, ovule-, carpel-, embryo-, endosperm-, pollen-, or flowers; a promoter for inducing expression in the presence of a plant hormone, such as auxins, gibberellins, cytokinins or brassinosteroids; or a promoter for expressing a gene during a particular developmental stage in a plant, such as during fruit ripening; or a constitutive promoter. The present invention also encompasses a recombinant construct including such a polynucleotide sequence.

In a second aspect, the present invention entails an isolated polypeptide comprising the cytochrome P450 polypeptide which when expressed in a plant produces a plant with at least one phenotype selected from the group consisting of parthenocarpic fruit and enlarged fruit.

The invention also encompasses a transgenic plant comprising an isolated polynucleotide comprising a nucleotide sequence encoding a cytochrome P450 polypeptide which when expressed in a plant produces a plant with at least one phenotype selected from the group consisting of parthenocarpic fruit and enlarged fruit when compared with a plant lacking said isolated polynucleotide. In one embodiment, the transgenic plant expresses an isolated cytochrome P450 polypeptide comprising (a) SEQ ID No. 1; (b) a sequence comprising conservative substitutions to SEQ ID No. 1; (c) a homologous sequence which when expressed in a plant produces parthenocarpic fruit or enlarged fruit; and (d) a sequence comprising a fragment of (a), (b) or (c) and produces parthenocarpic fruit or enlarged fruit. In a second embodiment, the transgenic plant expresses the isolated polynucleotide comprising SEQ ID No. 2 or 3, a homologous sequence or a fragment thereof. Additionally, the polynucleotide sequence may comprise a tissue-specific or active promoter, particularly, a promoter active in fruit, ovules, carpels, embryo, endosperm, pollen or flowers; a promoter for inducing expression in the presence of a plant hormone, such as auxins, gibberellins, cytokinins or brassinosteroids; or a promoter for expressing a gene during a particular developmental stage in a plant, such as during fruit ripening; or a constitutive promoter. The present invention also encompasses recombinant constructs including such a polynucleotide sequence.

In another aspect the invention encompasses a method for screening one or more compounds to identify a compound that controls parthenocarpy or fruit size in a plant. The method comprises introducing the compound into plant material; and monitoring the effect of the introduced compound on the expression or activity of a polypeptide selected from the group consisting of (i) SEQ ID No. 1; (ii) a sequence comprising conservative substitutions to SEQ ID No. 1; (iii) a homologous sequence which when expressed in a plant produces parthenocarpic fruit or enlarged fruit; and (iv) a sequence comprising a fragment of (i), (ii) or (iii), or the expression of a polynucleotide encoding the same.

In yet another aspect, the invention encompasses a method for producing a plant having at least one phenotype selected from the group consisting of parthenocarpic fruit and enlarged fruit. The method comprises expressing an isolated polynucleotide encoding a cytochrome P450 polypeptide in a plant; and selecting progeny with parthenocarpic or enlarged fruit. In one embodiment the cytochrome P450 polypeptide is selected from the group consisting of: (a) SEQ ID No. 1; (b) a sequence comprising conservative substitutions to SEQ ID No. 1; (c) a homologous sequence which when expressed in a plant produces parthenocarpic fruit or enlarged fruit; and (d) a sequence comprising a fragment of (a), (b) or (c) which when expressed in a plant produces parthenocarpic fruit or enlarged fruit. In a second embodiment, the method further comprises contacting the plant with a plant hormone, such as an auxin, gibberellin, cytokinin or brassinosteroid. In a third embodiment, the method further comprises pollinating the plant that expresses the isolated polynucleotide encoding the cytochrome P450 gene. The pollen may be isolated from (a) a fertile plant lacking the isolated polynucleotide encoding the cytochrome P450 gene or (b) a fertile plant comprising the isolated polynucleotide encoding the cytochrome P450 gene.

In yet a further aspect, the present invention entails a method for propagating a plant that is male- sterile. The method comprises introducing an activator component and a P450 component into two different plants to generate first and second transgenic plants. The activator component comprises a promoter operably linked to a transactivation factor for expression of the transactivation factor in a plant. The P450 component comprises an isolated polynucleotide comprising a nucleotide sequence encoding a cytochrome P450 polypeptide operably linked to a promoter for binding the transactivation factor and which does not express the P450 nucleotide sequence except for in the presence of the transactivation factor. Then first and second transgenic plants are crossed to generate a hybrid plant and a hybrid plant is selected that produces male-sterile plants.

Fruit crops and vegetables to be made seedless, or to increase fruit size or yields include, but are not limited to, melons, berries, peppers, tomatoes, citrus fruits, plums, alfalfa, squash, eggplant, peas, cotton, avocados, mangos, papayas, nectarines, apples, grapes, pears, peaches and cereals, such as corn, wheat, rice, sorghum and barley.

### DETAILED DESCRIPTION OF THE INVENTION

### DEFINITIONS

To ensure a complete understanding of the invention, the following definitions are provided.

A “cytochrome P450 or P450 polypeptide” refers to an enzyme that contains a heme-binding region and that may catalyze at least one hydroxylation step in a biosynthetic pathway in a plant.

A “transgenic plant” refers to a whole plant as well as to seed, fruit, leafs, roots, other plant tissue, plant cells, protoplasts, callus or any other plant material, and progeny thereof. Transgenic plants are plants which contain isolated polynucleotides or polypeptides which are introduced into plants, for example by transformation. Transformation means introducing a nucleotide sequence in a plant in a manner to cause stable or transient expression of the sequence. This may be achieved by transfection with viral vectors, transformation with plasmid vectors or introduction of naked DNA by electroporation, lipofection, particle gun acceleration or the like.

An “isolated polynucleotide” is a nucleotide sequence that is not in its native state, for example, when it is separated from nucleotide sequences with which it typically is in proximity in a genome or is next to other nucleotide sequences with which it typically is not. The nucleotide sequence may comprise a coding sequence or fragments thereof, promoters, introns, enhancer regions, polyadenylation sites, translation initiation sites, reporter genes, selectable markers or the like. The polynucleotide may be single stranded or double stranded DNA or RNA. The polynucleotide may be a genomic or processed nucleotide sequence (such as cDNA or mRNA). The nucleotide sequence may be in a sense or antisense orientation.

A “homologous sequence” means a sequence has a certain degree of sequence identity with a second sequence after alignment as determined by using sequence analysis programs for database searching and sequence comparison available from the Wisconsin Package, such as BLAST, FASTA, PILEUP, FINDPATTERNS or the like (GCG, Madison, Wiss.). Public sequence databases such as GENBANK, EMBL, Swiss-Prot and PIR or private sequence databases such as PhytoSeq (Incyte Pharmaceuticals, Palo Alto, Calif.) may be searched. Typically, homologous sequences when expressed in a plant may cause essentially the same effect, for example, two polypeptides having essentially the same effect on the number of seeds in the fruit of a plant or the size of the fruit of a plant.

An “isolated polypeptide” is a polypeptide derived from the translation of an isolated polynucleotide or that is more enriched than the polypeptide in its natural state in a cell, i.e. more than 5% enriched or 10% enriched.

A “fragment”, as applies to polypeptides, is a portion of a polypeptide that can perform at least one biological activity of the intact polypeptide in substantially the same manner as the intact polypeptide does. A fragment may vary in size from as few as 9 amino acids to the length of the intact polypeptide, but are preferably at least 30 amino acids in length. The amino acids selected from the intact polypeptide need not be consecutive. Exemplary fragments include fragments including amino acid residues 301-309 of SEQ ID No. 1, 319-336 of SEQ ID No. 1, 385-405 of SEQ ID No. 1, 415-427 of SEQ ID No. 1, 463-477 of SEQ ID No. 1, or combinations thereof. In reference to nucleotide sequences “a fragment” refers to any sequence of at least 15 nucleotides, preferably 50 nucleotides, more preferably at least 90 nucleotides, of any of the sequences provided herein and as an example include nucleotides 1-100, 101-200, 201-300, 501-600, 801-900, 1000-1015, or 1101-1300 of SEQ ID No. 2. SEQ ID No. 2 is an illustration of a fragment of SEQ ID No. 3.

A “fruit” refers to any seed-containing organ of a plant. In the case of cereals, each seed is a single-seeded fruit.

An “enlarged plant” refers to a plant which may have either larger fruit, larger stems, larger leafs, larger flowers or any combination of the above. The tissue of the enlarged plant is at least 5% larger and preferably at least 20% larger than that of the wild type plant. Of particular interest are plants having enlarged fruit that are at least 20% larger and preferably which are 40% larger.

A “parthenocarpic fruit” is a fruit with less seed than the wild type fruit, such as with at least 20% less seed and preferably with at least 50% less seed or is seedless.

A “promoter” is a polynucleotide sequence that controls the expression of a gene and is operably linked to a gene of interest. Constitutive promoters express a gene in all tissues, at all times and under all conditions. Specific promoters (or active promoters) may cause preferential (for example higher levels of expression in specific tissue, but not to the exclusion of lower expression levels in other tissue) or selective expression (for example levels of expression occur only under specific conditions to the exclusion of other expression) in particular tissue, at different developmental stages, or in response to endogenous or exogenous compounds. Expression levels of a transcript may be detected by Northerns, RT-PCR or gene expression array systems.

Taking into account these definitions, the present invention provides a means to control seed development in fruit or to control fruit size in a plant. Those skilled in the art will recognize that the present invention can be used in conjunction with virtually any plant or any cell, in particular plant cells.

The present invention relates to polynucleotide and polypeptide sequences for a P450 belonging to the CYP78 subfamily. These sequences may be employed for producing parthenocarpic fruit or for increasing fruit size, including vegetable or grain size, stem size, leaf size or flower size. An important and valuable use of increasing plant tissue size is to increase agricultural yields of plants. Additionally the sequences may be employed to produce male-sterile plants.

1. P450 Gene Identification

We have discovered in Arabidopsis thaliana a dominant gain of function mutation that causes, in the original genetic background (homozygous for the mutation apetala2-1), sterile fruits that are wider and flatter than is found without the new mutation. The plants also have larger stems, leafs, and flowers and are male-sterile plants. Normally, Arabidopsis fruits will not develop when ovules are not fertilized. In this mutant the fruits can reach a nearly normal size despite the failure of fertilization. The mutant was identified using a T-DNA activation-tagging screen of Arabidopsis thaliana plants. The T-DNA activation tagging screen entails transforming a plant with a gene tag containing multiple transcriptional enhancers and once the tag has inserted in the genome, expression of flanking DNA becomes deregulated and the mutation becomes dominant (Ichikawa et al., Nature 390 698-701 (1997), Kakimoto et al., Science 274: 982-985 (1996).

A 7.8 kb region of the genome causing the new mutant phenotype was cloned by plasmid rescue of the plant DNA flanking the T-DNA insertion. In the DNA flanking the right border, about 2 kilobase pairs from the right border of the T-DNA insertion, is a gene that encodes a cytochrome P450 gene. The cloned region of the 7.8 kb genome containing the P450 gene that is overexpressed is shown in SEQ ID No. 3. This genomic sequence when isolated and linked to the CaMV 35S promoter and expressed in plants produces seedless fruits.

A nucleotide sequence with introns excised corresponding to the mRNA species is shown in SEQ ID No. 2. In situ hybridization experiments have shown that, in wild-type Arabidopsis thaliana plants, the P450 gene is expressed at the RNA level in the funiculus of developing ovules. In the overexpressing, parthenocarpic line, the gene is expressed in the carpel valves, especially in the inner side of the carpel valves. SEQ ID No. 2 encodes a polypeptide of 534 amino acids. Amino acid sequence comparisons using the tblastn sequence analysis program showed that the identified sequence was homologous to members of the CYP78 family of plant cytochrome P450s. Homologous sequences were found both in the orchid Phalaenopsis sp. ‘hybrid SM9108’, the legume Glycine max (soybean), and in the cereal Zea mays (maize), indicating that related genes are present throughout flowering plants. Therefore, either the Arabidopsis gene or homologous gene sequences when expressed in a plant provides seedless fruit in most species of transgenic plants including monocots, dicots and gymnosperms. These genes, or homologous sequences identified in other or the same organism, may be used to transform plants to decrease seed production, increase fruit, stem, leaf or flower size and consequently increase yields for both vegetables and crops. Certain fragments are of particular interest because they belong to highly conserved regions of the identified homologs, and may be implicated in function. These fragments may be combined with other sequences to improve the biological activity of the polypeptide. The amino acid fragment sequences comprise:

(1) DFVDVLL (S/G)L,

(2) D(M/I)(V/I)A(I/V)LWEM(IV)FRGTDT(V/T)A,

(3) VKE(A/T/V)LR(L/A/M)HPPGPLLSWARL(A/S)(I/T),

(4) (I/V)PAGTTAMVN(M/T)W(A/S) or

(5) RLAPFG (A/S) G(R/K) R (V/I/A) CPGK.

We have also discovered that when plants expressing these sequences are pollinated large fruits with seed are produced. Pollination may occur by cross-pollination or self-pollination. Pollination may occur when the sequence is expressed from either constitutive or tissue specific promoters, such as the DefH9 promoter (Rotino et al. (1997) Nature Biotech. 15: 1398-1401) or using a carpel active promoter such as AGL5 (Savidge et al. (1995) Plant Cell 7:721-33), AGL8 (Mandel et al. (1995) Plant Cell 7:763-71) and AGL 1(Yung et al. (1999) Plant J 17:203-8) and AGL13 (Rounsley et al. (1995) Plant Cell 7:1259-69). Self-pollination occurs when gene expression is directed from a tissue-specific promoter.

It is known that plants with ovule-specific expression of an auxin biosynthetic enzyme can produce parthenocarpic fruit (Rotino et al. Nature Biotechnology (1997) 15: 1398-1401). It has recently been demonstrated that overexpression of the gibberellin (GA) 20-oxidase results in GA overproduction in plants (Shihshieh et al., (1998) Plant Physiology 118: 773-781). GA overproduction in plants also may result in parthenocarpic fruit. Expression of these sequences or other sequences implicated in plant hormone biosynthetic pathways, or the topical administration of these hormones, may be combined with the expression of the P450 gene to decrease seed production or increase fruit size. Also, the expression of the P450 gene itself may be placed under the control of a hormone inducible promoter so that P450 expression and plant hormone overexpression occur in a plant at the same time.

2. Methods For Detecting Homologous Sequences

Homologous sequences (homologs) identified in Arabidopsis thaliana or in other plants may also be used to change the phenotype of plants and in particular that of fruit. Homologs may be derived from agriculturally important plant species, including but not limited to, crops such as soybean, wheat, corn, potato, cotton, rice, oilseed rape (including canola), sunflower, alfalfa, sugarcane and turf; or fruits and vegetables, such as banana, blackberry, blueberry, strawberry, and raspberry, cantaloupe, carrot, cauliflower, coffee, cucumber, eggplant, grape, honeydew, lettuce, mango, melon, onion, papaya, peas, peppers, pineapple, spinach, squash, sweet corn, tobacco, tomato, watermelon, rosaceous fruits (such as apple, peach, pear, cherry and plum) and vegetable brassicas (such as broccoli, cabbage, cauliflower, brussel sprouts and kohlrabi). Other crops, fruits and vegetables whose seed's phenotype may be changed include barley, currant, avocado, citrus fruits such as oranges, lemons, grapefruit and tangerines, artichoke, cherries, nuts such as the walnut and peanut, endive, leek, roots, such as arrowroot, beet, cassava, turnip, radish, yam, sweet potato and beans.

P450s that are homologs of the disclosed polypeptide sequences will typically share at least 40% amino acid sequence identity. More closely related P450s may share at least 50%, 60%, 65%, 70%, 75% or 80% sequence identity with the disclosed sequences. Factors that are most closely related to the disclosed sequences share at least 85%, 90% or 95% sequence identity. At the nucleotide level, the sequences will typically share at least 40% nucleotide sequence identity, preferably at least 50%, 60%, 70% or 80% sequence identity, and more preferably 85%, 90%, 95% or 97% sequence identity.

Homologs from the same plant, different plant species or other organisms may be identified using database sequence search tools, such as the Basic Local Alignment Search Tool (BLAST) (Altschul et al. (1990) Basic local alignment search tool. J. Mol. Biol. 215:403-410; and Altschul et al. (1997) Gapped BLAST and PSI-BLAST: A New Generation of Protein Database Search Programs Nucleic Acid Res.25: 3389-3402). Several sequence analysis programs (blastp, blastn, blastx, tblastp, tblastn and tblastx) are available from several sources, including GCG (Madison, Wis.) and the National Center for Biotechnology Information (NCBI, Bethesda, Md.). When using the sequence analysis program tblastn, the BLOSUM-62 scoring matrix (Henikoff, S. and Henikoff, J. G. (1992) Proc. Natl. Acad. Sci. USA 89: 10915-10919) may be employed. Sequences with the highest scores and an exemplary cutoff E value threshold for tblastn less than −70, and preferably less than −100, are identified as homologous sequences.

Substitutions, deletions and insertions introduced in the sequences are also envisioned by this invention. Amino acid substitutions are typically of single residues; insertions usually will be on the order of about from 1 to 10 amino acid residues; and deletions will range about from 1 to 30 residues. Deletions or insertions preferably are made in adjacent pairs, i.e., a deletion of two residues or insertion of two residues. Substitutions, deletions, insertions or any combination thereof may be combined to arrive at a final construct. Obviously, the mutations that are made in the DNA encoding the protein must not place the sequence out of reading frame and preferably will not create complementary regions that could produce secondary mRNA structure.

Substitutions are those in which at least one residue in the amino acid sequence has been removed and a different residue inserted in its place. Such substitutions generally are made in accordance with the following Table 1 when it is desired to finely modulate the characteristics of the protein. Table 1 shows amino acids which may be substituted for an amino acid in a protein and which are typically regarded as conservative substitutions.

TABLE 1

| Residue | Conservative Substitutions |
|---|---|
| Ala | ser |
| Arg | lys |
| Asn | gln; his |
| Asp | glu |
| Gln | asn |
| Cys | ser |
| Glu | asp |
| Gly | pro |
| His | asn; gln |
| Ile | leu, val |
| Leu | ile; val |
| Lys | arg; gln; glu |
| Met | leu; ile |
| Phe | met; leu; tyr |
| Ser | thr; gly |
| Thr | ser; val |
| Trp | tyr |
| Tyr | trp; phe |
| Val | ile; leu |

Substitutions that are less conservative than those in Table 1 may be selected by selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine.

Homologous sequences also encompass polypeptide sequences that are modified by chemical or enzymatic means. Modifications include acetylation, carboxylation, phosphorylation, glycosylation, modified amino acids and the like. Protein modification techniques are illustrated in Ausubel et al. (eds) Current Protocols in Molecular Biology, John Wiley & Sons (1998).

An alternative indication to show whether two nucleic acid molecules are closely related is that the two molecules hybridize to each other under stringent conditions. Stringent conditions are sequence dependent and are different under different environmental parameters. Generally, stringent conditions are selected to be about 5° C. to 20° C. lower than the thermal melting point (Tm) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found in Sambrook et al., Molecular Cloning. A Laboratory Manual, Ed. 2, Cold Spring Harbor Laboratory Press, New York (1989)) and Tijssen (1993) Laboratory Techniques in Biochemistry and Molecular Biology—Hybridization with Nucleic Acid Probes Part I, Chapter 2 “Overview of principles of hybridization and the strategy of nucleic acid probe assays”, Elsevier, N.Y. (1993). Nucleic acid molecules that hybridize under stringent conditions will typically hybridize to a probe based on either the entire cDNA or selected portions of the cDNA under wash conditions of 0.2×SSC to 2.0×SSC, 0.1% SDS at 55-65° C., for example 0.2×SSC, 0.1% SDS at 65° C.

For conventional hybridization the hybridization probe is conjugated with a detectable label such as a radioactive label, and the probe is preferably of at least 20 nucleotides in length. As is well known in the art, increasing the length of hybridization probes tends to give enhanced specificity. The labeled probe derived from the Arabidopsis nucleotide sequence may be hybridized to a plant cDNA or genomic library and the hybridization signal detected using means known in the art. The hybridizing colony or plaque (depending on the type of library used) is then purified and the cloned sequence contained in that colony or plaque isolated and characterized.

The degeneracy of the genetic code further widens the scope of the present invention as it enables major variations in the nucleotide sequence of a DNA molecule while maintaining the amino acid sequence of the encoded protein. Overall, P450s that are homologs of the disclosed sequences will typically share at least 30% nucleotide sequence identity with a homologous sequence. More closely sequences may share at least 50%, 60%, 65%, 70%, 75% or 80% sequence identity with the disclosed nucleotide sequences. P450 s that are most closely related to the disclosed nucleotide sequences share at least 85%, 90% or 95% sequence identity with one or more of the disclosed Arabidopsis P450 proteins.

Homologs of the Arabidopsis P450s may alternatively be obtained by immunoscreening an expression library. With the provision herein of the disclosed P450 nucleic acid sequences, the polypeptide may be expressed and purified in a heterologous expression system (e.g., E. coli) and used to raise antibodies (monoclonal or polyclonal) specific for the P450. Antibodies may also be raised against synthetic peptides derived from P450 amino acid sequences. Methods of raising antibodies are well known in the art and are described in Harlow and Lane (1988) Antibodies: A Laboratory Manual, Cold Spring Harbor Laboratory, New York. Such antibodies can then be used to screen an expression library produced from the plant from which it is desired to clone the P450 DNA homolog, using the methods described above. The selected cDNAs can be confirmed by sequencing and enzymatic activity.

3. Recombinant Constructs

Any of the identified sequences may be incorporated in a recombinant construct for expression in plants. A number of recombinant vectors suitable for stable transformation of plant cells or for the establishment of transgenic plants have been described including those described in Weissbach and Weissbach, (1989) Methods for Plant Molecular Biology, Academic Press, and Gelvin et al., (1990) Plant Molecular Biology Manual, Kluwer Academic Publishers. Specific examples include those derived from a Ti plasmid of Agrobacterium tumefaciens, as well as those disclosed by Herrera-Estrella, L., et al., Nature 303: 209 (1983), Bevan, M., Nucl. Acids Res. 12: 8711-8721 (1984), Klee, H. J., Bio/Technology 3: 637-642 (1985) for dicotyledonous plants.

Alternatively, non-Ti vectors can be used to transfer the DNA into monocotyledonous plants and plant cells by using free DNA delivery techniques. Such methods may involve, for example, the use of liposomes, electroporation, microprojectile bombardment, silicon carbide wiskers, viruses and pollen. By using these methods transgenic plants such as wheat, rice (Christou, P., Bio/Technology 9: 957-962 (1991)) and corn (Gordon-Kamrn, W., Plant Cell 2: 603-618 (1990)) are produced. An immature embryo can also be a good target tissue for monocots for direct DNA delivery techniques by using the particle gun (Weeks, T. et al., Plant Physiol. 102: 1077-1084 (1993); Vasil, V., Bio/Technology 10: 667-674 (1993); Wan, Y. and Lemeaux, P., Plant Physiol. 104: 37-48 (1994), and for Agrobacterium-mediated DNA transfer (Hiei et al., Plant J. 6: 271-282 (1994); Rashid et al., Plant Cell Rep. 15: 727-730 (1996); Dong, J., et al., Mol. Breeding 2: 267-276 (1996); Aldemita, R. and Hodges, T., Planta 199: 612-617 (1996); Ishida et al., Nature Biotech. 14: 745-750 (1996)).

Typically, plant transformation vectors include one or more cloned plant genes (or cDNAs) under the transcriptional control of 5′ and 3′ regulatory sequences and a dominant selectable marker. Such plant transformation vectors typically also contain a promoter (e.g., a regulatory region controlling inducible or constitutive, environmentally-or developmentally-regulated, or cell- or tissue-specific expression), a transcription initiation start site, a ribosome binding site, an RNA processing signal, a transcription termination site, and/or a polyadenylation signal.

Examples of constitutive plant promoters which may be useful for expressing the P450 sequence include: the cauliflower mosaic virus (CaMV) 35S promoter, which confers constitutive, high-level expression in most plant tissues (see, e.g., Odel et al., (1985) Nature 313:810); the nopaline synthase promoter (An et al., (1988) Plant Physiol. 88:547); and the octopine synthase promoter (Fromm et al., (1989) Plant Cell 1: 977).

A variety of plant gene promoters that regulate gene expression in response to environmental, hormonal, chemical, developmental signals, and tissue also can be used for expression of the P450 sequence in plants, as illustrated seed-specific promoters (such as the napin, phaseolin or DC3 promoter described in U.S. Pat. No. 5,773,697), fruit-specific promoters that are active during fruit ripening (such as the dru 1 promoter (U.S. Pat. No. 5,783,393), or the 2A11 promoter (U.S. Pat. No. 4,943,674) and the tomato polygalacturonase promoter (Bird et al. (1988) Plant Mol. Biol. 11:651), pollen-active promoters such as PTA29, PTA26 and PTA13 (U.S. Pat. No. 5,792,929), promoters active in vascular tissue (Ringli and Keller (1998) Plant Mol. Biol. 37:977-988), flower-specific (Kaiser et al, (1995) Plant Mol. Biol. 28:231-243), pollen (Baerson et al. (1994) Plant Mol. Biol. 26:1947-1959), carpels (Ohl et al. (1990) Plant Cell 2:837-848), pollen and ovules (Baerson et al. (1993) Plant Mol. Biol. 22:255-267), auxin-inducible promoters (such as that described in van der Kop et al (1999) Plant Mol. Biol. 39:979-990 or Baumann et al. (1999) Plant Cell 11:323-334), cytokinin-inducible promoter (Guevara-Garcia (1998) Plant Mol. Biol. 38:743-753), promoters responsive to gibberellin (Shi et al. (1998) Plant Mol. Biol. 38:1053-1060, Willmott et al. (1998) 38:817-825) and the like.

Plant transformation vectors may also include RNA processing signals, for example, introns, which may be positioned upstream or downstream of the open reading frame sequence. In addition, the expression vectors may also include additional regulatory sequences from the 3′-untranslated region of plant genes, e.g., a 3′ terminator region to increase mRNA stability of the mRNA, such as the PI-II terminator region of potato or the octopine or nopaline synthase 3′ terminator regions.

Finally, as noted above, plant transformation vectors may also include dominant selectable marker genes to allow for the ready selection of transformants. Such genes include those encoding antibiotic resistance genes (e.g., resistance to hygromycin, kanamycin, bleomycin, G418, streptomycin or spectinomycin) and herbicide resistance genes (e.g., phosphinothricin acetyltransferase).

A reduction of P450 activity in a transgenic plant to obtain smaller fruit or plants with more seed may be obtained by introducing into plants antisense constructs based on the P450 cDNA. For antisense suppression, the P450 cDNA is arranged in reverse orientation relative to the promoter sequence in the transformation vector. The introduced sequence need not be the full length P450 cDNA or gene, and need not be exactly homologous to the P450 cDNA or gene found in the plant type to be transformed. Generally, however, where the introduced sequence is of shorter length, a higher degree of homology to the native P450 sequence will be needed for effective antisense suppression. Preferably, the introduced antisense sequence in the vector will be at least 30 nucleotides in length, and improved antisense suppression will typically be observed as the length of the antisense sequence increases. Preferably, the length of the antisense sequence in the vector will be greater than 100 nucleotides. Transcription of an antisense construct as described results in the production of RNA molecules that are the reverse complement of mRNA molecules transcribed from the endogenous P450 gene in the plant cell. Suppression of endogenous P450 gene expression can also be achieved using ribozymes. Ribozymes are synthetic RNA molecules that possess highly specific endoribonuclease activity. The production and use of ribozymes are disclosed in U.S. Pat. No. 4,987,071 to Cech and U.S. Pat. No. 5,543,508 to Haselhoff. The inclusion of ribozyme sequences within antisense RNAs may be used to confer RNA cleaving activity on the antisense RNA, such that endogenous mRNA molecules that bind to the antisense RNA are cleaved, which in turn leads to an enhanced antisense inhibition of endogenous gene expression.

Constructs in which RNA encoding the P450 cDNA (or homologs thereof) is over-expressed may also be used to obtain co-suppression of the endogenous P450 gene in the manner described in U.S. Pat. No. 5,231,020 to Jorgensen. Such co-suppression (also termed sense suppression) does not require that the entire P450 cDNA be introduced into the plant cells, nor does it require that the introduced sequence be exactly identical to the endogenous P450 gene. However, as with antisense suppression, the suppressive efficiency will be enhanced as (1) the introduced sequence is lengthened and (2) the sequence similarity between the introduced sequence and the endogenous P450 gene is increased.

Constructs expressing an untranslatable form of the P450 gene may also be used to suppress the expression of endogenous P450 activity. Methods for producing such constructs are described in U.S. Pat. No. 5,583,021 to Dougherty et al. Preferably, such constructs are made by introducing a premature stop codon into the P450 gene.

4. Transgenic Plants with Modified P450 Expression

Once a construct comprising a nucleotide sequence encoding a P450 gene of this invention has been isolated, standard techniques may be used to express the cDNA in plants in order to modify that particular seed characteristic.

Exemplary plants to be transformed may be any higher plant, including monocotyledonous and dicotyledenous plants. Suitable protocols are available for Leguminosae (alfalfa, soybean, clover, etc.), Umbelliferae (carrot, celery, parsnip), Cruciferae (cabbage, radish, rapeseed, broccoli, etc.), Curcurbitaceae (melons and cucumber), Gramineae (wheat, corn, rice, barley, millet, etc.), Solanaceae (potato, tomato, tobacco, peppers, etc.), and various other crops. See protocols described in Ammirato et al. (1984) Handbook of Plant Cell Culture—Crop Species. Macmillan Publ. Co. Shimnamoto et al. (1989) Nature 338:274-276; Fromm et al. (1990) Bio/Technology 8:833-839; and Vasil et al. (1990) Bio/Technology 8:429-434.

Transformation and regeneration of both monocotyledonous and dicotyledonous plant cells is now routine, and the selection of the most appropriate transformation technique will be determined by the practitioner. The choice of method will vary with the type of plant to be transformed; those skilled in the art will recognize the suitability of particular methods for given plant types. Suitable methods may include, but are not limited to:

electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumeficiens (AT) mediated transformation.

Successful examples of the modification of plant characteristics by transformation with cloned cDNA sequences which serve to illustrate the current knowledge in this field of technology, and which are herein incorporated by reference, include: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,610,042.

Following transformation, plants are preferably selected using a dominant selectable marker incorporated into the transformation vector. Typically, such a marker will confer antibiotic or herbicide resistance on the transformed plants, and selection of transformants can be accomplished by exposing the plants to appropriate concentrations of the antibiotic or herbicide.

After transformed plants are selected and grown to maturity, they can be assayed using the methods described herein to determine whether P450 activity has been altered as a result of the introduced recombinant polynucleotide, such as by analyzing mRNA expression using Northern blots or microarrays, or by visual inspection of plant seed or biochemical assays.

After establishing that the transformed plants do overexpress the P450 gene, the plants may be used to isolate an endogenous plant growth chemical that affects fruit and seed size, and yields in plants. The large fruits, stems, leafs or flowers of the transformed plants are harvested and the chemicals present in them fractionated by standard fractionation into organic phases and water-soluble fractions. These fractions are assayed for bioactivity on immature siliques of Arabidopsis in culture. The active fractions that produce larger siliques are further purified and sufficient material is obtained to identify the structure of the hormonally produced chemical in the transformed plants. Identified chemicals may be useful for spraying on fruit, vegetable and grain crops to increase fruit, vegetable and grain sizes and yields.

Additionally, plants or plant material expressing the P450 gene may be employed for screening other compounds that may control parthenocarpy or fruit, stem, leaf or flower size in a plant. The method entails first introducing a compound into the plant or a host cell. The compound may be introduced by topical administration of the exogenous compound and then monitoring the effect of the exogenous compound on the expression of the P450 polypeptide or the expression of the polynucleotide encoding the same so as to detect changes in expression. Changes in the expression of the P450 polypeptide may be monitored by use of polyclonal or monoclonal antibodies, two-dimensional polyacrylamide electrophoresis (2D-PAGE) or the like. Changes in the expression of the corresponding polynucleotide sequence may be detected by use of microarrays, Northerns or any other technique for monitoring changes in mRNA expression. These techniques are exemplified in Ausubel et al. (eds) Current Protocols in Molecular Biology, John Wiley & Sons (1998).

Furthermore the present invention can be a method for propagating a plant that is male-sterile. The method comprises producing a first transgenic plant comprising an activator component comprising a promoter operably linked to a transactivation factor. In one embodiment, the promoter is active in carpels and the transactivation factor is a LacI/Gal4 transactivation factor. Additionally a second transgenic plant is produced comprising the P450 gene operably linked to a promoter for binding the transactivation factor and which does not express P450 gene except for in the presence of the transactivator factor. In this case a suitable binding site for the LacI/Gal4 transactivation factor is multiple LacI binding sites. Neither transgenic plant overexpresses the P450 gene and therefore both plants have fertile seeds. However, once the two plants are crossed to produce hybrid plants that contain both the activator component and the P450 gene, the resulting hybrid plants will overexpress the P450 gene and the hybrid plant is male-sterile. Such a system is generally described in Guyer et al. (1998) Genetics 149:633-639.

The following examples are provided to better elucidate the practice of the present invention and should not be interpreted in any way to limit the scope of the present invention. Those skilled in the art will recognize that various modifications, truncations, etc. can be made to the methods and genes described herein while not departing from the spirit and scope of the present invention.

### EXAMPLES

The identification of sequences implicated in parthenocarpy and increasing the size of various plant materials, vector construction, plant transformation and the observed phenotype of transgenic plants expressing the sequences are described in the following examples.

### Example 1

### Soil Mix Preparation and Plant Growth

If not otherwise indicated, the soil mix was prepared by mixing 4 scoops of soil (RodMcLellan Co., San Mateo, Calif.) with 3 scoops of vermiculite (THERM-O-ROCK, Chandler, Ariz.) and 2 scoops of perlite (THERM-O-ROCK) and approximately 500 ml of water. Optionally, a very thin layer (0.25 cm) of Redi-earth soilless mix was added. 9-11 white pots were placed in a tray. The tray was imbibed with 3.5 liters water with 20 ml gnatrol and 5ml of Ortho Daconil Fungicide. The surface of the soil was sprayed with water before planting. Planting 18 seeds per pot (3 rows of 6) gave reasonably good growth. After planting, the tray was covered with Saran Wrap, taping it to the tray, so that air holes remain. This keeps the humidity high, encouraging germination. The tray was placed in a cold room for at least 3 days (5-7 days optimal). Plants germinated after 3-4 days. After germination was complete, the Saran Wrap was removed. While the plants were young, i.e. up to the 4-5 leaf stage, the soil was kept moist. After that the soil was allowed to partially dry out periodically. Plants were grown under continuous illumination at about 500-1000 fc fluorescent light (cool white).

### Example 2

### Activation Tagging of the P450 Gene

Arabidopsis thaliana (Landsberg erecta ecotype) apetala 2-1 mutants were transformed with the pSKI15 vector available from the Weigel Laboratory at The Salk Institute (Weigel et al. http:/biosun.salk.edu/LABS!pbio-w/). The pSK115 plasmid contains multimerized CaMV 35S enhancers and the bar gene which confers Basta resistance and is derived from pPCVICEn4HPT (Hayashi et al. Science 258: 1350-1353, Walden et al. Plant Mol. Biol 26: 1521-1528). Plants were transformed by a vacuum infiltration method (Bechtold et al., C. R. Acad. Sci. Paris, Life Sciences 316: 1194-1199 (1993)). Transgenic plants were grown and selected for Basta resistance (D'Halluin et al. Meth. in Enzymol. 216 415-427 (1992)). Pots were prepared by putting cheesecloth at the bottom and adding sand to the height of 4 cm. The pots were soaked in the selection buffer (1.22 g Hoagland's No.2 BASAL SALT mixture, 37.5 microliters BASTA (600 g/liter)/3 liters). The seeds were planted at high density in the pots. The plants were vernalized for 3 days in a cold room, and then moved to a growth chamber. After about 10 days, transformants were transferred to soil mix in pots.

A dominant gain of function mutant was characterized in the original genetic background (homozygous for the mutation apetala2-1), sterile fruits that are wider and flatter than is found without the new mutation. Normally, Arabidopsis fruits will not develop when ovules are not fertilized. In this mutant the fruits can reach a nearly normal size, despite failure of fertilization.

The mutant also has short petals, short stamens, is male sterile, and shows reduced female fertility. When the apetala2-1 mutation is crossed out of the genetic background, elongated rather than wide fruits are obtained. In this genetic background the plants are almost female sterile, and petals, while delayed in elongation, can be of normal size.

### Example 3

### Southern Analysis

Genomic DNA was first purified using the CTAB mini prep method. The leaf tissue was ground in liquid nitrogen and extracted in 2×CTAB buffer (3% CTAB (hexadecyltrimethylammonium bromide)), 100 mM Tris-HCl pH 8.0, 20 mM EDTA, 1.4 M NaCl, 0.2% beta mercaptoethanol). The tubes were incubated for 30 minutes at 60° C. An equal volume of chloroform was added. The tubes were spun at 9000 rpm for 10 minutes. The upper layer was saved. Genomic DNA was precipitated by adding ⅔ volume isopropanol. The pellet was rinsed with 75% ethanol. The pellet was resuspended in TE, including 20 microliters/ml RNase. The tubes were incubated at 37° C. for 30 minutes. ½ volume 7.5 M ammonium acetate was added and the tubes were extracted with 1 volume of chloroform. The supernatant was saved. Genomic DNA was precipitated with 2.5 times total volumes of ethanol. The pellet was washed, dried, and resuspended in 50 microliters TE.

For Southern hybridization experiments, ³²P-labelled probe (2-3 million cpm/ml) were hybridized with the hybridization buffer (6×SSC, 5×Denhardt's, 0.1% SDS, 100 micrograms/ml denatured salmon sperm DNA) at 65° C. The probe was the 35S enhancer region (339 nucleotides long) which was excised from the pSKI15 vector using EcoRV. Southern blot analysis using the enhancer fragment of the 35S promoter as a probe showed that a single insertion in a 6.2 kb genomic fragment after digestion with EcoRI caused the mutation and was not observed in the wild type.

### Example 4

### Northern Analysis

To isolate RNA, a leaf sample was placed in a liquid nitrogen-filled Eppendorf tube and ground using a plastic pestle (Kimble/Kontes, Vineland, N.J.). 1 ml TRI reagent (Molecular Research Center, Inc., Cincinnati, Ohio) was added. The solution was mixed by vortexing and stood for 10 minutes at room temperature. 200 microliters chloroform were added. The tubes were mixed vigorously and let stand for 5 minutes at room temperature. The tubes were spun at 14,000 rpm for 15 minutes. The aqueous phase was removed. 0.25 ml isopropanol and 0.25 ml buffer (0.8M sodium citrate, 1.2M NaCl) were added. The tubes stood for 10 minutes at room temperature. The tubes were then centrifuged for 15 minutes and rinsed with 75% ethanol. The pellet was dried and resuspended in water.

For Northern blot analysis, 10 micrograms of total RNA were loaded onto a gel (300 mls)(260 ml water, 30 ml 10×MSE buffer (200 mM MOPS, 50 mM sodium acetate, 10 mM EDTA), 3.6 g agarose, 9.0 ml formaldehyde). After electrophoresis, the RNA was transferred to a Hybond-NX membrane (Amersham Pharmacia Biotech, Piscataway, N.J.) and hybridized with each probe. For hybridization, ³²P-labeled probe (2-3 million cpm/ml) were hybridized with the hybridization buffer (50% formamide, 6×SSC, 5×Denhardt's, 0.1% SDS, 100 micro g/ml denatured salmon sperm DNA) at 42° C. The probe was the flanking region cloned by plasmid rescue using KpnI. A 3.5 kb fragment was excised using EcoRI from the plasmid and used as a probe. A strong signal was observed only from the transgenic callus not from the wild type callus.

### Example 5

### The Cloned Genomic Region Produces Pathenocarpic Fruits

The 7.8 kb genomic region causing the new mutant phenotype was cloned by plasmid rescue of the plant DNA flanking the T-DNA insertion using the restriction enzyme KpnI. The fragment contains the four tandemly repeated 35S enhancer elements, 2 kb upstream promoter region of the gene and the coding region of the P450 gene. This chimeric gene was transformed into wild-type Arabidopsis thaliana plants using a T-DNA vector system. The transgenic plants reproduced the phenotypes of the original mutant line in a wild-type genetic background: short stamens, male sterile, reduced female fertility, and elongated seedless carpels. Thus the 7.8 kb genomic fragment that includes the P450 gene is sufficient to cause parthenogenic fruit development.

The plasmid rescue protocol is described below. About 0.3˜1.0 micrograms purified genomic DNA were digested with KpnI for two hours at 37 ° C. The digestion products were phenol extracted, chloroform extracted and ethanol precipitated. The pellet was washed with with 70% ethanol, dried, and resuspended in 360 microliters double distilled water. Additionally, 40 microliters 10×ligation buffer, 4 microliters 100 mM ATP, 1 microliter T4 DNA ligase (1 u) were added and incubated overnight at 16 ° C. To quench the reaction 40 microliters 3 M sodium acetate, 1 microgram yeast t-RNA, and 1000 microliters ethanol were added and mixed and left to sit for 10 minutes at room temperature. Then the precipitate was spun down for 10 minutes. The pellet was washed with 70% ethanol, dried and resuspended in 3 microliters double distilled water.

Electroporation was performed using the Gene Pulser II electroporator (BioRad, Hercules, Calif.). 1 mm cuvettes and cuvette holder were cooled on ice. The settings for electroporation were as follows voltage: 1.8 kV, capacitor: 25 microFaradays, resistor: 200 ohms. Frozen competent cells (Electro Max DH10B cells, Life Technologies, Inc., Rockville, Md.) were thawed on ice. 20 microliters of the cell solution were added to 1 microliter of ligated DNA in an Eppendorf tube and incubated on ice for 30 to 60 seconds. Cells were transferred to the cuvette and pulsed. Immediately thereafter 1 ml SOC medium was added, the cell suspension was transferred to a 17×100 mm polypropylene tube and shaken at 37° C. for 1 hour. 10 microliters were spread on a carbenicillin plate. The concentration of carbenicillin on the E. coli selection plates was 100 micrograms/ml. The remaining cells were transferred to an Eppendorf tube, spun 5 k for 1 minute, 900 microliters of the SOC medium were removed and the remaining solution spread on the carbenicillin plate.

### Example 6

### Transformation of Agrobacterium with Expression Vector Containing SEQ ID No. 2 or 3

Agrobacterium ASE strain cells (40 microliters) were mixed with 50-100 ng DNA (generally resuspended in 10 mM Tris-HCl, 1 mM EDTA, pH 8.0) and incubated on ice for 30 to 60 seconds. The DNA/cell mixture was then transferred to a chilled cuvette with a 2 mm electrode gap and subject to a 2.5 kV charge dissipated at 25 μF using a Gene Pulser II apparatus (BioRad, Hercules Calif.). After electroporation, cells were immediately resuspended in 1.0 ml LB and allowed to recover without antibiotic selection for 90 minutes at 28° C. in a shaking incubator. After recovery, cells were plated onto selective medium (LB broth containing 100 μg/ml spectinomycin (Sigma)) and incubated for 24-48 hours at 28° C. Single colonies were then picked and inoculated in fresh medium. The presence of the plasmid construct was verified by PCR amplification and sequence analysis.

### Example 7

### Transformation of Arabidopsis Plants with Agrobacterium tumefaciens with Expression Vector

Seeds were planted in a pot. After plants bolted, the primary inflorescent shoot was clipped off to encourage the growth of secondary inflorescent shoots. Infiltration was performed 4 to 8 days after clipping. A liquid culture of Agrobacterium tumefaciens (ASE strain) carrying the construct was grown. 5 ml overnight culture was started two days ahead of infiltration. One day prior to infiltration the culture was used to inoculate a 400 ml culture. After 24 hours of growth, cells were usually at a density of at least 1 OD. The cells were harvested by centrifugation and resuspended to an OD of 0.8 in infiltration media. Using tupperware the plants were dipped in infiltration solution (5×MS salts, 1×B5 vitamins, 5% sucrose, 0.044 micromolar benzylamino purine, 0.03% Silwet L-77 (OSI Specialties, Inc. Danbury Conn.) and put into a vacuum oven at room temperature under pressure at 10-15 in³ Hg for 10-15 minutes. After the vacuum was released, pots were removed, laid on their side in a tray and covered with Saran wrap. The next day the Saran wrap was removed and the pots were placed upright.

T1 seeds collected from the vacuum-infiltrated T0 plants were sterilized in 10% bleach, 0.02% Triton X-100. The seeds were rinsed 3-4 times with sterile water. The sterilized seeds were plated by resuspending in 0.1% agarose at room temperature and pipeting the seeds onto selection plates (B5 medium, 0.8% Bacto-agar, 50 microgram/ml kanamycin). 2000-4000 seed were plated per 150×15 mm plate. The plants were vernalized for three nights in a cold room at 4° C. The plates were moved to growth chamber. After about 7 days, transformants that showed dark green color with long roots were transferred to soil.

### Example 8

### Pollination with Wild-type Pollen Produces Very Large Fruit

The flowers to be crossed were marked by tying cotton thread at the peduncle. By using forceps (INOX No.5; Fontax, Electron Microscopy Sciences, Fort Washington, Pa.) washed with 70% ethanol, stamens of wild type flower in bloom were pulled out. The pistil was pollinated by tapping the stigmatic organs of P450 transgenic plant with the open side of the tapetum of wild type stamens. After pollination with wild-type pollen of the P450 overexpression line, the fertilized plants were found to produce siliques that could be at least 40% larger and up to 600% larger than normal and that were seeded. Thus the combination of overexpression of the P450 gene and fertilization with wild-type pollen has been found to be particularly effective at producing very large fruits which are seeded.

Self-pollination with fertile pollen may also result in large fruit, for example, by the use of a carpel active promoter such as AGL5 (Savidge et al. (1995) Plant Cell 7:721-33), AGL8 (Mandel et al. (1995) Plant Cell 7:763-71) and AGL11 (Yung et al. (1999) Plant J 17:203-8) and AGL13 (Rounsley et al. (1995) Plant Cell 7:1259-69) or an ovule specific promoter such as the DefH9 promoter (Rotino et al. Nature Biotechnology (1997) 15: 1398-1401) to express the P450 gene.

### Example 9

### Transformation of Cereal Plants with Plasmid Vectors Containing SEQ ID No. 2 or 3

Cereals can also be transformed with the plasmid vectors containing the sequence and constitutive or tissue-specific promoters The tissue specific promoters may include aleurone specific promoters, embryo specific promoters such as globulin 1, and endosperm specific promoters such as the maize 27 kd zein promoter and the rice glutelin 1 promoter. In these cases, the cloning vector, pMEN020, is modified to replace the NptII coding region with the BAR gene of Streptomyces hygroscopicus that confers resistance to phosphinothricin. The KpnI and BglII sites of the Bar gene are removed by site-directed mutagenesis with silent codon changes.

It is now routine to produce transgenic plants of most cereal crops (Vasil, I., Plant Molec. Biol. 25: 925-937 (1994)) such as corn, wheat, rice, sorghum (Cassas, A. et al., Proc. Natl. Acad Sci USA 90: 11212-30 11216 (1993) and barley (Wan, Y. and Lemeaux, P. Plant Physiol. 104:37-48 (1994) Other direct DNA transfer methods such as the microprojectile gun or Agrobacterium tumefaciens-mediated transformation can be used for corn (Fromm. et al. Bio/Technology 8: 833-839 (1990)); wheat (Vasil et al., Bio/Technology 11:1553-1558 (1993, rice (Hiei et al., Plant Mol Biol. 35:205-18 (1997)).

Plasmids according to the present invention may be transformed into corn embryogenic cells derived from immature scutellar tissue by using microprojectile bombardment, with the A188XB73 genotype as the preferred genotype (Fromm, et al., Bio/Technology 8: 833-839 (1990)). After microprojectile bombardment the tissues are selected on phosphinothricin to identify the transgenic embryogenic cells (Gordon-Kamm et al., Plant Cell 2: 603-618 (1990)). Transgenic plants are regenerated by standard corn regeneration techniques.

### Example 10

### Identification of Homologs

Homologs from the same plant, different plant species or other organisms were identified using the Basic Local Alignment Search Tool (BLAST) (Altschul et al. (1997) Gapped BLAST and PSI-BLAST: A New Generation of Protein Database Search Programs Nucleic Acid Res. 25: 3389-3402) (NCBI, Bethesda, Md.). Tblastn compares a polypeptide query sequence with all 6 open reading frames of database nucleotide sequences. GENBANK sequence databases were searched. The E value threshold for tblastn was less than −100. SEQ ID No. 1 was shown to be homologous to the sequences shown in Table 2. The annotation for the different sequence hits and the sequence identity when compared with the P450 polypeptide sequences are presented.

TABLE 2

| SEQ ID | | Amino Acid Sequence |
|---|---|---|
| Nos. | Annotation | Identity |
| 4 and 5 | mRNA from A. thaliana chromosome | 80.6% |
| | II BAC T3A4 genomic sequence | |
| | (g4415928) | |
| 6 and 7 | Glycine max cytochrome P450 | 66% |
| | monooxygenase mRNA (g2739008) | |
| 8 and 9 | Pinus radiata cytochrome P450 | 54% |
| | (PRE74) mRNA (g2935524) | |
| 10 and 11 | Zea mays cytochrome P450 (cyp78) | 47% |
| | mRNA (g349717) | |
| 12 and 13 | Phalaenopsis sp. ‘hybrid SM9108’ | 54% |
| | cytochrome p450 mRNA (g1173623) | |

### Example 11

### Synergistic interaction of P450 Overexpression with Plant Hormone Overproducing Plants

The combination of overexpression of the P450 gene with an auxin overproducing line such as that described in Rotino et al. Nature Biotechnology (1997) 15: 1398-1401 should result in large seedless fruit. The auxin overproducing line has a construct comprising a MADS-box from Antirrhinum majus for selective expression in ovules and the iaaM gene from Pseudomonas syringae which sythesizes indoleacetamide, an intermediate in the biosynthesis of indole-3-acetic acid. The auxin overproducing line may be generated by introducing into Agrobacterium tumefaciens a recombinant plasmid, based on the binary vector pMEN020. A chimeric DefH9-iaaM gene, carried on the vector includes the promoter region and untranslated signal regions, including an intron, from the DefH9 gene from Antirrhinum majus, the coding region of the iaaM gene and terminator sequences from the nopaline synthase gene of A. tumefaciens. The transgenic plant overexpressing the P450 gene is cotransformed with a similar vector using a carpel active promoters such as AGL5 (Savidge et al. (1995) Plant Cell 7:721-33), AGL8 (Mandel et al. (1995) Plant Cell 7:763-71) and AGL11 (Yung et al. (1999) Plant J 17:203-8) and AGL13 (Rounsley et al. (1995) Plant Cell 7:1259-69) to generate plants overexpressing both P450 and iaaM gene.

The combination of overexpression of the P450 with a gibberellin (GA) overproducing line (Shihshieh et al. (1998) Plant Physiology 118: 773-781) will result in large seedless fruit. The GA 20-oxidase gene expression would be expressed from an ovule specific promoter such as that taught in Example 9. The use of a flower specific or carpel enhanced promoter to express the P450 gene should result in sterile flowers that produce large seedless fruits.

### Example 12

### Synergistic Interaction of P450 Overexpression with the Exogenous Adminstration of Plant Hormones

Transgenic seeds are germinated on Petri plates containing nutrient medium (Wilson et al. (1990) Mol. Gen. Genet. 222:377-383) containing 1% agarose and 1% sucrose and 0.1 micromolar 2,4-dichlorophenoxy acetic acid (2,4-D), a synthetic auxin. Alternatively, the hormone may have been administered by spraying (1 micromolar 2,4-D) or soaking the plant with the hormone. These 2,4-D treated plants will produce large seedless fruits.

The above examples are provided to illustrate the invention but not to limit its scope. Other variations of the invention will be readily apparent to one of ordinary skill in the art and are encompassed by the appended claims. All publications, patents and patent applications cited herein are hereby incorporated by reference.

We claim:

1. An isolated polynucleotide comprising a nucleotide sequence encoding a cytochrome P450 polypeptide which when expressed in a plant produces at least one phenotype selected from the group consisting of parthenocarpic fruit and enlarged fruit.

2. The polynucleotide of claim 1, wherein said cytochrome P450 polypeptide comprises a sequence selected from the group consisting of:
(a) SEQ ID No. 1;
(b) a sequence comprising one or more conservative substitutions to SEQ ID No. 1;
(c) a homologous sequence which when expressed in a plant produces parthenocarpic fruit or enlarged fruit; and
(d) a sequence comprising a fragment of (a), (b) or (c) which when expressed in a plant produces parthenocarpic fruit or enlarged fruit.

3. The polynucleotide of claim 1, wherein said cytochrome P450 polypeptide comprises a sequence selected from the group consisting of (a) a sequence comprising amino acids 301-309, 319-336, 385-405, 415-427, or 463-477 of SEQ ID No. 1; and (b) conservative substitutions to said sequence.

4. The polynucleotide of claim 1, wherein the nucleotide sequence comprises a sequence selected from the group consisting of SEQ ID No. 2 or 3, a homologous sequence or a fragment thereof.

5. The polynucleotide of claim 1, further comprising a tissue-specific promoter.

6. The polynucleotide of claim 5, wherein said tissue-specific promoter is selected from the group consisting of fruit, ovule-, carpel-, embryo-, endosperm-, pollen-, and flower-specific promoters.

7. The polynucleotide of claim 1; further comprising a promoter for expressing a gene in the presence of a plant hormone.

8. The polynucleotide of claim 8, wherein said plant hormone is selected from the group consisting of auxins, gibberellins, cytokinins and brassinosteroids.

9. The polynucleotide of claim 1, further comprising a promoter for expressing a gene during fruit ripening.

10. The polynucleotide of claim 1, further comprising a constitutive promoter.

11. An isolated polynucleotide comprising a nucleotide sequence encoding a cytochrome P450 polypeptide comprising a sequence selected from the group consisting of (a) a sequence comprising amino acids 301-309, 319-336, 385-405, 415-427, and 463-477 of SEQ ID No. 1; and (b) conservative substitutions to said sequence.

12. A recombinant construct comprising the polynucleotide of claim 1.

13. An isolated polypeptide comprising a cytochrome P450 polypeptide which when expressed in a plant produces a plant with at least one phenotype selected from the group consisting of parthenocarpic fruit and enlarged fruit

14. The polypeptide of claim 13, wherein said cytochrome P450 polypeptide comprises a sequence selected from the group consisting of:
(a) SEQ ID No. 1;
(b) a sequence comprising one or more conservative substitutions to SEQ ID No. 1;
(c) a homologous sequence which when expressed in a plant produces parthenocarpic fruit or enlarged fruit; and
(d) a sequence comprising a fragment of (a), (b) or (c) which when expressed in a plant produces parthenocarpic fruit or enlarged fruit.

15. The polypeptide of claim 13, wherein said cytochrome P450 polypeptide comprises a sequence selected from the group consisting of (a) a sequence comprising amino acids 301-309, 319-336, 385-405, 415-427, or 463-477 of SEQ ID No. 1; and (b) conservative substitutions to said sequence.

16. A transgenic plant comprising an isolated polynucleotide comprising a nucleotide sequence encoding a cytochrome P450 polypeptide which when expressed in a plant produces a plant with at least one phenotype selected from the group consisting of parthenocarpic fruit and enlarged fruit when compared with a plant lacking said isolated polynucleotide.

17. The transgenic plant of claim 16, wherein said cytochrome P450 polypeptide comprises a sequence selected from the group consisting of: (a) SEQ ID No. 1; (b) a sequence comprising conservative substitutions to SEQ ID No. 1; (c) a homologous sequence which when expressed in a plant produces parthenocarpic fruit or enlarged fruit; and (d) a sequence comprising a fragment of (a), (b) or (c) which when expressed in a plant produces parthenocarpic fruit or enlarged fruit.

18. The transgenic plant of claim 16, wherein the nucleotide sequence is SEQ ID No. 2 or 3, homologous sequences or fragments thereof.

19. The transgenic plant of claim 16, further comprising a tissue-specific promoter.

20. The transgenic plant of claim 19, wherein said tissue- specific promoter is a fruit, ovule-, carpel-, embryo-, pericarp-, endosperm-, pollen-, flower-specific promoter.

21. The transgenic plant of claim 16, further comprising a promoter for expressing a gene in the presence of a plant hormone.

22. The transgenic plant of claim 21, wherein said plant hormone is selected from the group consisting of auxins, gibberellins, cytokinins and brassinosteroids.

23. The transgenic plant of claim 16, further comprising a promoter specific for expressing a gene during fruit ripening.

24. The transgenic plant of claim 16, further comprising a constitutive promoter.

25. The transgenic plant of claim 19, further comprising a polynucleotide for increasing the level of an endogenous plant hormone.

26. The transgenic plant of claim 23, wherein said plant hormone is selected from the group consisting of auxins, gibberellins, cytokinins and brassinosteroids.

27. A transgenic plant which expresses a cytochrome P450 polypeptide comprising a sequence selected from the group consisting of: (a) SEQ ID No. 1; (b) a sequence comprising conservative substitutions to SEQ ID No. 1; (c) a homologous sequence which when expressed in a plant produces parthenocarpic fruit or enlarged fruit; and (d) a sequence comprising a fragment of (a), (b) or (c), wherein said transgenic plant has at least one phenotype selected from the group consisting of parthenocarpic fruit and enlarged fruit.

28. A method for screening one or more compounds to identify a compound that controls parthenocarpy or fruit size in a plant, said method comprising (a) introducing the compound into the plant; and (b) monitoring the effect of the introduced compound on the expression or activity of a polypeptide selected from the group consisting of (i) SEQ ID No. 1; (ii) a sequence comprising conservative substitutions to SEQ ID No. 1; (iii) a homologous sequence which when expressed in a plant produces parthenocarpic fruit or enlarged fruit; and (iv) a sequence comprising a fragment of (i), (ii) or (iii) which when expressed in a plant produces parthenocarpic fruit or enlarged fruit, or the expression of a polynucleotide encoding the same.

29. A method for producing a plant having a modified phenotype, said method comprising: (a) expressing an isolated polynucleotide encoding a cytochrome P450 polypeptide in a plant; and (b) selecting the plant with the modified phenotype.

30. The method of claim 29, wherein said cytochrome P450 polypeptide is selected from the group consisting of: (a) SEQ ID No. 1; (b) a sequence comprising conservative substitutions to SEQ ID No. 1; (c) a homologous sequence which when expressed in a plant produces parthenocarpic fruit or enlarged fruit; and (d) a sequence comprising a fragment of (a), (b) or (c), wherein said transgenic plant has parthenocarpic fruit or enlarged fruit.

31. The method of claim 29, further comprising contacting said plant with a plant hormone.

32. The method of claim 27, wherein said plant hormone is selected from the group consisting of auxins, gibberellins, cytokinins and brassinosteroids.

33. The method of claim 27, further comprising pollinating the plant that expresses the isolated polynucleotide encoding the cytochrome P450 gene.

34. The method of claim 31, wherein said pollinating step comprises using pollen from (a) a fertile plant lacking the isolated polynucleotide encoding the P450 gene or (b) a fertile plant comprising the isolated polynucleotide encoding the P450 gene.

35. A method for propagating a plant that is male-sterile, said method comprising: (a) introducing an activator component comprising a promoter operably linked to a transactivation factor for expression of the transactivation factor into a first plant to produce a first transgenic plant; (b) introducing a P450 component comprising an isolated polynucleotide comprising a nucleotide sequence encoding a cytochrome P450 polypeptide operably linked to a promoter for binding the transactivation factor and which does not overexpress the nucleotide sequence except for in the presence of the transactivator factor into a second plant to produce a second transgenic plant; (c) crossing said first and second transgenic plants to generate a hybrid plant; and (d) selecting a hybrid plant that is male-sterile.
